(19) United States
(12) Patent Application Publication
(10) Pub. No.: US 2026/0321374 A1
(43) Pub. Date: Oct. 15, 2026

(54) DETERMINING CORRELATION STRENGTH OF FACTORS ON SYMPTOM EXPRESSION IN HEALTH CONDITIONS USING FACTOR TIME

(71) Applicant: Shiny New App, LLC, Minneapolis, MN (US)

(72) Inventor: Lynn Smith, Minneapolis, MN (US)

(21) Appl. No.: 18/998,733

(22) PCT Filed: Jul. 25, 2023
(86) PCT No.: PCT/US2023/070912

§ 371 Date: Jan. 27, 2025

Related U.S. Application Data
(60) Provisional application No. 63/392,524, filed on Jul. 27, 2022.

(51) Int. Cl. G16H 20/00

(52) U.S. Cl. CPC G16H 20/00

(57) ABSTRACT

A method includes providing an interface, receiving first user data via the interface, the first user data indicating occurrence of one or more factor events and time information for each of the one or more factor events, receiving second user data via the interface, the second user data indicating occurrence of one or more symptom expressions and time information for each of the one or more symptom expressions, storing the first and second user data, determining correlation strength using the first and second user data, the correlation strength being based on the time information of the one or more factor events and the time information of the one or more symptom expressions, and outputting the correlation strength to the interface. The correlation strength may be a ranking listing the one or more factor events having the greatest correlation with the one or more symptom expressions of a health condition.

### CROSS-REFERENCE TO RELATED APPLICATION

This application claims priority to U.S. Provisional Patent Application Ser. No. 63/392,524, titled PATTERN FINDING FOR EPISODIC CONDITIONS, filed on Jul. 27, 2022, the disclosure of which is herein incorporated by reference in its entirety.

### TECHNICAL FIELD

The present disclosure relates generally to devices, systems, and methods for determining correlation strength between factors and symptom expressions of a health condition. More specifically, embodiments of the present disclosure can collect times of factor events and times of symptom expressions to determine correlation strength between a factor and a symptom expression.

### BACKGROUND

The typical approach to determining correlation strength for a factor that may contribute to or cause a change in migraine expression starts by recording a time associated with a migraine and recording the presence or lack of presence of a factor. This approach then repeats this process by recording one or more subsequent migraines in a similar manner. This creates a set of migraines. In this typical approach, documentation of factors relating to migraines are recorded in a binary manner as part of the migraine record that indicates, or is used to determine, that the “factor was present” or the “factor was not present.” This typical approach could then use the number of times the factor was present in the migraine set in an evaluation that determines correlation strength. This typical approach then outputs the correlation strength as a score, ranking or other indication, or uses the correlation strength in other evaluations that are then output.

For example, in this typical approach, if a user records four migraines and specifies three instances of having a factor present, the evaluation system could determine a correlation based on the number of times the factor was present as a ratio to the number of times a migraine was recorded. In this typical approach, the time that the instance of the factor occurred is neither recorded nor used in evaluating the correlation. As such, in the noted example, the typical approach could determine that a particular event (e.g., eating cheese) had a ¾, or 75%, correlation to the onset of a migraine, which could be considered a strong correlation. In this example, an evaluation system uses a simple ratio to determine correlation strength.

However, this typical approach can be inaccurate for several reasons. As one example, in many instances, the aggregation of the number of events of a potential factor type in comparison to the aggregate number of migraine occurrences may not allow for evaluation beyond ratio-based algorithms or algorithms that are simplistic. Without collecting and using information related to the timing of factors, determining which potential factor is more highly correlated with inducing symptom or episode expression changes could be inaccurate.

### SUMMARY

Various embodiments disclosed herein relate to determining correlation strength between each of one or more potential factors and symptom expressions of a health condition. In particular, certain such embodiments could record a set of factor events and symptom events and use these events to determine correlation strength between one or more factor events and changes in symptom expression. To attempt to provide a more accurate correlation assessment for how a factor correlates to a symptom expression, such embodiments can start by documenting a set of factor events and a set of symptom events. Accordingly, such embodiments can use an evaluation system that discerns one or more patterns that correlate to the result of a change in the occurrences of the symptom expressions. In embodiments, evaluation of the episodic condition could include using an absolute or implied elapsed time between factor events and symptom events. Certain such embodiments could evaluate a set of factor event times and a set of symptom expression times with an evaluation system to determine correlation strength (e.g., correlation or relevance score) of each such potential types of triggering factors.

Embodiments of the disclosure include a method performing operations stored in a non-transitory computer readable medium, the operations including providing an interface for receiving data, optionally directly from a user, receiving first user data via the interface, the first user data indicating, at least, occurrence of one or more factor events and time information for each of the one or more factor events, receiving second user data via the interface, the second user data indicating, at least, occurrence of one or more symptom expressions of a health condition and time information for each of the one or more symptom expressions of a health condition, storing the first user data and the second user data in a storage unit, determining correlation strength using the first user data and the second user data, the correlation strength being based the time information for each of the one or more factor events and the time information for each of the one or more symptom expressions of a health condition, and outputting the correlation strength to the interface, optionally as a ranking between the one or more factor events and the one or more symptom expressions of a health condition, the ranking listing one or more factors correlated with the health condition in descending order based on correlation strength (i.e., a factor or more than one factor can be ranked based on correlation strength with the health condition; the ranking is determined using the first user data and the second user data). The first user data and the second user data can be received and stored separately in any order or received and stored together.

Embodiments of the disclosure include a system including a user interface and a processing unit and a storage unit, the processing unit and the storage unit configured to perform operations including receiving first user data via the user interface, the first user data indicating, at least, occurrence of one or more factor events and time information for each of the one or more factor events, receiving second user data via the user interface, the second user data indicating, at least, occurrence of one or more symptom expressions of a health condition and time information for each of the one or more symptom expressions of a health condition, storing the first user data and the second user data in a storage unit, determining correlation strength using the first user data and the second user data, the correlation strength being based on the time information for each of the one or more factor events and the time information for each of the one or more symptom expressions of a health condition, and outputting the correlation strength to the user interface, optionally as a ranking between the one or more factor events and the one or more symptom expressions of a health conditions, the ranking listing the one or more factor events having the greatest correlation with the one or more symptom expressions of a health condition in descending order based on correlation strength, wherein the first user data and the second user data can be received and stored separately in any order or received and stored together.

Embodiments of the disclosure include a non-transitory storage article for performing an evaluation of correlation strength between one or more factor events and one or more symptom expressions of a health condition, including non-transitory computer-executable instructions for providing an interface for receiving data, optionally directly from a user, receiving first user data via the interface, the first user data indicating, at least, occurrence of one or more factor events and time information for each of the one or more factor events, receiving second user data via the interface, the second user data indicating, at least, occurrence of one or more symptom expressions of a health condition and time information for each of the one or more symptom expressions of a health condition, storing the first user data and the second user data in a storage unit, determining correlation strength using the first user data and the second user data, the correlation strength being based on the time information for each of the one or more factor events and the time information for each of the one or more symptom expressions of a health condition, and outputting the correlation strength to the interface, optionally as a ranking between the one or more factor events and the one or more symptom expressions of a health condition, the ranking listing the one or more factor events having the greatest correlation with the one or more symptom expressions of a health condition in descending order based on correlation strength, wherein the first user data and the second user data can be received and stored separately in any order or received and stored together.

The above summary is not intended to describe each illustrated embodiment or every implementation of the subject matter hereof. The figures and the detailed description that follow more particularly exemplify various embodiments.

The differentiated techniques of embodiments disclosed herein gathering a set of times associated with instances of one or more factor types and using the set of times along with a set of times associated with symptom expression in analysis, as compared to the typical aggregation approach explained previously, can provide several useful advantages. As one example, the techniques disclosed herein can help to provide a more accurate assessment of the cause, or causes, of health condition events, and in particular episodic health condition events, and thus provide a user with a better ability to address and reduce, or prevent, future instances of the health condition event. For instance, in cases where a user has one or more factor types affecting the symptom expression of their chronic condition, the techniques disclosed herein can provide an improved ability to more accurately determine which factor type or factor types have a stronger or weaker correlation to symptom expression changes in individuals. Notably, the approach of the techniques disclosed herein can make the strength of a correlation between factor types and symptom changes more clear which, in turn, can facilitate more accurate, more effective, and more efficiency in addressing and reducing, or preventing, future instances of the episodes of an episodic condition.

### BRIEF DESCRIPTION OF THE DRAWINGS

Subject matter hereof may be more completely understood in consideration of the following detailed description of various embodiments in connection with the accompanying figures, in which:

FIG. 1 illustrates a user interface for inputting one or more potential trigger events and one or more episodic conditions, in accordance with embodiments of the disclosure.

FIG. 2A illustrates a user interface for displaying correlation strength, in accordance with embodiments of the disclosure.

FIG. 2B illustrates a user interface for displaying correlation strength, in accordance with embodiments of the disclosure.

FIG. 3 illustrates a user interface for inputting one or more potential trigger events and one or more episodic conditions, in accordance with embodiments of the disclosure.

FIG. 4 illustrates a user interface for a permission to record data element, in accordance with embodiments of the disclosure.

FIG. 5 illustrates a flow diagram of a method of determining correlation strength between at least one potential trigger event and a subsequently occurring episodic condition, in accordance with embodiments of the disclosure.

FIG. 6 illustrates a system for carrying out a method of determining correlation strength between at least one potential trigger event and a subsequently occurring episodic condition, in accordance with embodiments of the disclosure.

While various embodiments are amenable to various modifications and alternative forms, specifics thereof have been shown by way of example in the drawings and will be described in detail. It should be understood, however, that the intention is not to limit the claimed inventions to the embodiments described. On the contrary, the intention is to cover all modifications, equivalents, and alternatives falling within the spirit and scope of the subject matter as defined by the claims.

### DEFINITIONS

A Set of Events—two or more events
Correlation—a mutual relationship or connection between two or more things (e.g., a factor event and a symptom expression of a health condition); any correlation metric including a covariance matrix, a regression, standard deviation, a combination thereof, or another statistical indicator or indicator of association
Elapsed Time—time difference or time interval between a factor and an episodic condition; can be explicitly determined (e.g., calculated using an algorithm or other suitable method) or implicitly determined (e.g., algorithm or other suitable method uses factor time and episodic condition time to determine correlation strength). Implicit determination of elapsed time may be performed using an artificial intelligence (AI) system where, for example, a table of time values are provided to a neural network and a correlation is determined based on a time interval corresponding to the table of time values
Episodic Condition—any disorder that includes the appearance of one or more symptoms or factors in discrete, often brief, periods or episodes (e.g., migraines, food sensitivity, hives, mood disorder, epilepsy, hypertension, asthma, diabetes, major depressive disorder, bipolar disorder, schizophrenia, or other health conditions falling within the definition)
Evaluation System—a system or algorithm that evaluates input data; not limited to structured or unstructured data (e.g., a pattern finder algorithm or any algorithm or any machine learning/AI system/neural network). May include a processing unit of an electronic device that is configured to receive input data, include time information, and subsequently calculate and/or determine a correlation between a factor and an episodic condition or symptom expression of a health condition
Factor—something that could affect the human body (e.g., consumables such as food, drinks, or medicine; activities such as physical exercise, travel, talking; absence of certain activities such as lack of sleep, lack of exercise, dehydration)
Factor Event—the instance of an encounter with a factor that includes a time (e.g., eating chocolate on May 17, 2013, at 3:00 am, taking a plane flight on Friday afternoon, coffee on Tuesday, a week of vacation); the factor could affect the body, could not affect the body, or may be suspected of affecting the body
Health Condition—any condition affecting the body, including arthritis, joint disease, an auto-immune disorder, an immune-related disorder, an inflammatory disease, lupus, thyroid disease, gout, diabetes, chronic fatigue syndrome, insomnia, depression, a psychological disease, gastrointestinal disease, colitis, ulcerative colitis, inflammatory bowel disease, Crohn's disease, candida, celiac disease, irritable bowel syndrome, one or more food allergies, one or more food sensitivities, morning sickness, menstrual cramps, chronic pain, back pain, facial pain, fibromyalgia, asthma, migraines, abdominal migraines, cyclic vomiting syndrome, cluster headaches, chronic headaches, tension headaches, another type of headache, seizures, epilepsy, neurodermatitis, acne, psoriasis, adiposity, hypertonia, heart disease, hypertension, cardiovascular disease, arteriosclerosis, a form of cancer, and/or acquired immune deficiency syndrome, or combinations thereof
Symptom—a physical or mental feature regarded as indicating a condition of disease, particularly one that is apparent to the patient
Symptom Expression—a symptom existing, an amount of symptom, or a change to symptoms in the body. This could be a new symptom such as having a migraine. This could be at one point in time or over a span of time (e.g., headache level 5, having severe eye pain on Friday evening, frequency of headaches, having fewer episodes, severity of eye pain in June) (e.g., severity, length, frequency)
Time Information—any time or time interval recorded in any format. Time can be down to the second, minute, hour, day, or over longer periods of time. Time can also be recorded over a range such as “morning” or “afternoon,” or “first half of the day” or “second half of the day” (e.g., time interval corresponding to a factor and an episodic condition). May be a single specific time or more than one time value (e.g., 8:30 am on May 17, 2013; 9:00 am amount change; 9:45 am on May 17, 2013, as a start time and an end time)
Time-Based Event Data—data that includes time recorded to exactly or approximately the second, minute, hour, span of hours, day, period of time, time interval, or range of time

### DETAILED DESCRIPTION OF THE DRAWINGS

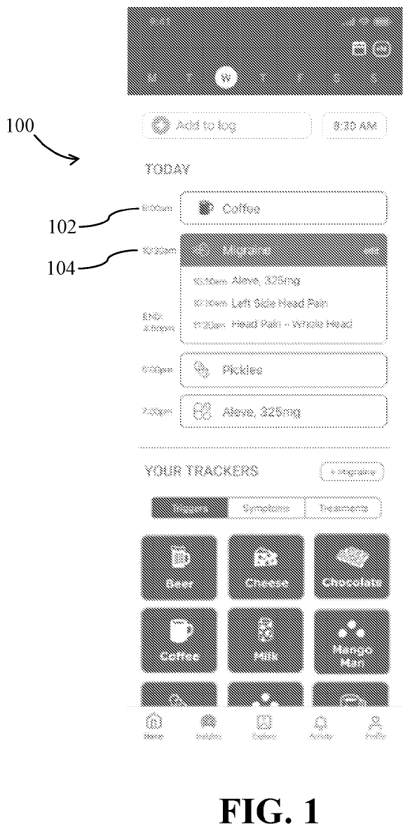

FIG. 1 illustrates an embodiment of a user interface 100. The user interface 100 can be configured for inputting one or more potential trigger events and inputting one or more episodic conditions. And, in response thereto, the user interface 100 can be configured for providing correlation strength (e.g., relevance score) of the potential trigger event to the occurrence of the episodic condition based on time information, for example elapsed time or time interval, between the occurrence of the one or more potential triggering events (also referred to herein as “factor events” or “factors”) and the occurrence of the one or more episodic conditions (also referred to herein as “symptom expressions of a health condition”).

The user interface 100 can be presented, for instance, at a computing device, such as a mobile computing device or another similar electronic device. The computing device can include programmable processing circuitry and a non-transitory storage article storing non-transitory computer-executable instructions. The non-transitory computer-executable instructions can be executed at the computing device by the programmable processing circuitry to cause the user interface 100 to display various items.

For example, the user interface 100 can include one or more regions, such as region 102, for inputting both a type of one or more potential triggering events, in this example drinking coffee, and a time of the occurrence of each of the one or more types of potential triggering events, in this example 8:00 am as an occurrence time for the potential triggering event of drinking coffee. Likewise, the user interface 100 can include one or more regions, such as region 104, for inputting both a type of episodic condition, in this example a migraine, and a time of the occurrence of the type of episodic condition, in this example 10:30 am as an occurrence time for the episodic condition of a migraine. The time of the occurrence of the type of potential triggering event and the time of the occurrence of the type of subsequent episodic condition can be associated with a start, end, midpoint, or other time relating to the occurrence of the type of potential triggering event and the occurrence of the type of subsequent episodic condition. In the illustrated example, the type of potential triggering event and the occurrence of a subsequent episodic condition can be limited to those occurring within a preset time period of one another, such as a twenty-four-hour (e.g., one day) time period, though in other examples the preset time period can vary, such as a function of the type of episodic condition being tracked.

As one example, the user interface 100 can be used to help discern one or more patterns between one or more preceding potential triggering events and a subsequently occurring episode of an episodic condition. The exemplary illustration of the user interface 100 at FIG. 1 shows the user interface 100 configured for helping to discern one or more patterns between one or more preceding potential triggering events and a migraine as the example of a subsequently occurring episode of a migraine episodic condition. The user interface 100 can use the input times of types of one or more potential triggering events and the input subsequent time of an occurrence of an episode of a type of episodic condition to display to the user a relative ranking of the strength of the correlation between a specific type of input potential triggering event and the type of subsequently occurring episode of a type of episodic condition. With respect to the illustrated example relating to a migraine as the type of subsequently occurring episode of a type of episodic condition, user interface 100 can output a correlation metric, such as a score or a line of text indicating a metric, that corresponds to a degree of correlation between each received potential triggering event from the user and the subsequently occurring migraine episode. The computer-executable instructions can check for several different patterns, in relation to the same input potential triggering event and/or in relation to different input potential triggering events. One or more of the patterns scored uses a determined or calculated timespan in finding the score. The overall score for a factor is the highest scoring pattern that was found. This can be shown to the user on a ranked list which provides the inputted trigger events having the greatest correlation with the subsequently occurring migraine episode in descending order based on correlation strength. The user can go into a detail screen that shows the correlation metric (e.g., score) for each type of pattern analysis that was used for each factor.

An output provided at the user interface 100 as to a relative ranking of the strength of the correlation between a specific type of input potential triggering event and the type of subsequently occurring episode of a type of episodic condition can vary in different embodiments. As one example, the user interface 100 can output a numerical score on a preset scale (e.g., a numerical score on a preset scale of zero to one hundred; e.g., eating a banana has a 43% correlation to a subsequent onset of the specified type of episodic condition). In a further such example, the user interface 100 can present each received type of potential triggering event in a ranked order where each displayed type of potential triggering event is ordered according to its score and relative to the score of other received types of potential triggering events. The correlation strength can be provided directly to a user or used as an input for further evaluation (e.g., when doing subsequent migraine prediction, knowing the correlation strength from previous factor events is an input for the subsequent migraine prediction).

Another, additional or alternative, type of output that can be provided at the user interface 100 can be a prediction as to when another episode of the episodic condition will occur in the future. Namely, the processing circuity can use the one or more input times of the one or more types of the one or more potential triggering events, as well as the input subsequent time of an occurrence of an episode of a type of episodic condition, to discern a pattern that may have resulted in the occurrence of the episode of a type of episodic condition. Then, the processing circuitry can use this discerned pattern to predict when another episode of that episodic condition will occur in the future. For example, where a previously received type of potential triggering condition included a time when a user was exposed to a relatively high degree of pollen content and a migraine occurred a relatively short amount of time thereafter, the processing circuitry, via execution of the computer-executable instructions, can output (e.g., at the user interface 100) an indication that a relatively high degree of pollen is expected to be present at a current or future time and the user is thus more likely to experience a migraine at that current or future time.

As another example, where another, different user's profile characteristics are similar to the present user's profile characteristics and that different user was able to reduce or eliminate future episodes of the particular, common episodic condition by switching from one intake (e.g., intake of one medication (e.g., Relax)) to another intake (e.g., intake of another, different medication (e.g., Imitrex)), the processing circuitry, via execution of the computer-executable instructions, can output (e.g., at the user interface 100) an indication that the present user should consider the same switch in intakes to likewise help reduce or eliminate future episodes of the particular, common episodic condition.

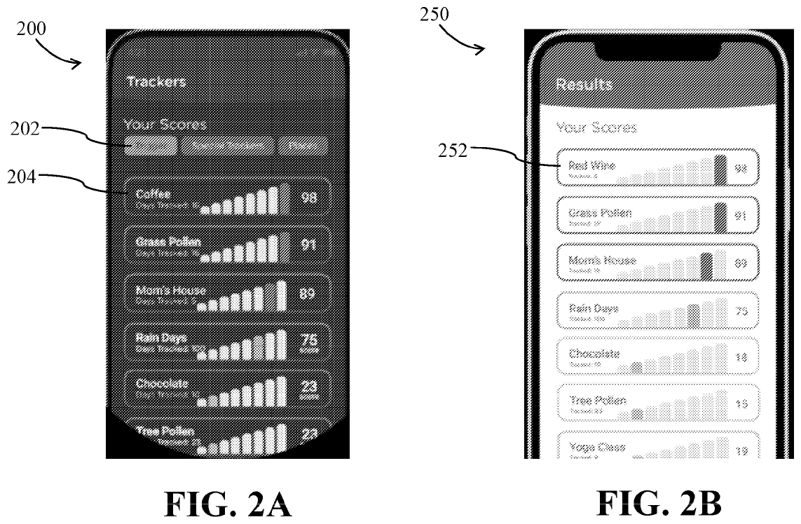

FIGS. 2A and 2B illustrate embodiments of user interface 200 and user interface 250, respectively. User interfaces 200/250 can be configured for outputting and/or displaying a ranking listing one or more factor events having the greatest correlation with one or more symptom expressions of a health condition in descending order based on correlation strength. User interfaces 200/250 can be presented, for instance, at a computing device, such as a mobile computing device or another similar electronic device, similar to user interface 100.

For example, user interfaces 200/250 can include one or more regions, such as region 202 and region 204 for user interface 200 and region 252 and region 254 for user interface 250. Region 202 may include one or more buttons for changing the display of user interface 200 from a ranking of triggers to special trackers (e.g., coffee, physical exercise) to places (e.g., work, gym, airport). Regions 204 and 252 may include, for example, an indication of a factor affecting the body being tracked, the number of days the factor has been tracked, a bar chart indicating relevance, and a numeric score indicating the correlation strength for that factor. It should be understood that user interfaces 200/250 can include several regions 204 and 252, respectively, for outputting and/or displaying several factors affecting the body that are ranked in descending order based on correlation strength (i.e., factor most correlated with a symptom expression of a health condition, or having the highest correlation strength, listed first; factor least correlated with a symptom expression of a health condition, or having the lowest correlation strength, listed last).

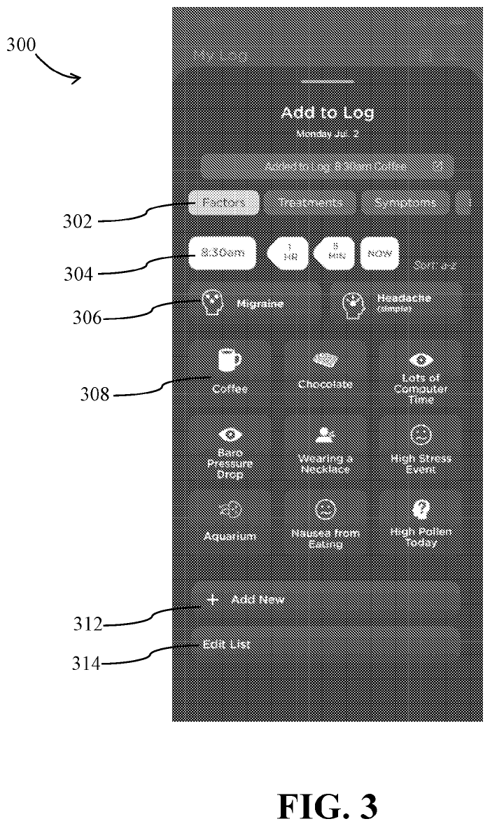

FIG. 3 illustrates a user interface 300 operationally similar to user interface 100. User interface 300 can be configured for inputting one or more potential trigger events and inputting one or more episodic conditions. And, in response thereto, the user interface 300 can be configured for providing correlation strength of the potential trigger event to the occurrence of the episodic condition based on an elapsed time between the occurrence of one or more factor events and the occurrence of one or more symptom expressions of a health condition. User interface 300 can be presented, for instance, at a computing device, such as a mobile computing device or another similar electronic device.

For example, the user interface 300 can include one or more regions, such as region 302, for changing the display from “factors” to “treatments” to “symptoms” and to other displays, region 304 for entering or receiving time information, region 306 for selecting a type of symptom expression of a health condition, such as “migraine” or “headache (simple),” region 308 for selecting a type of factor event that is currently being tracked, region 312 for adding a type of factor event to the list of factor events being tracked as shown in region 308, and region 314 for editing the list of factor events being tracked as shown in region 308.

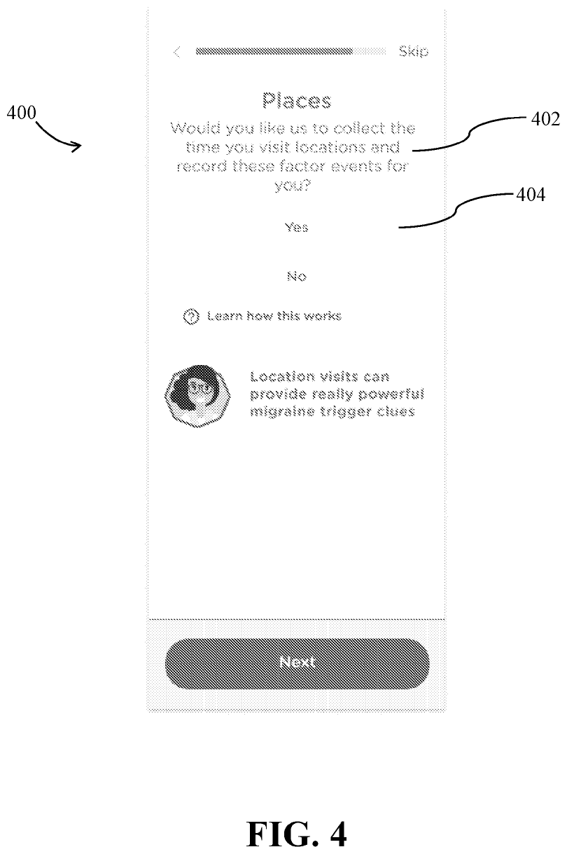

FIG. 4 illustrates a user interface 400 for a permission to record data element having one or more regions. For example, user interface 400 may include region 402 which presents a permission question, such as “Would you like us to collect the time you visit locations and record these factor events for you?” User interface 400 may also include region 404 which provides one or more buttons that a user can select to input an answer to the permission question presented in region 402. User interface 400 may include a progress dial which indicates how far a user has progressed through a series of questions, including application permission questions. User interface 400 may provide other regions for inputting information or for displaying relevant information, such as health condition resources, to a user.

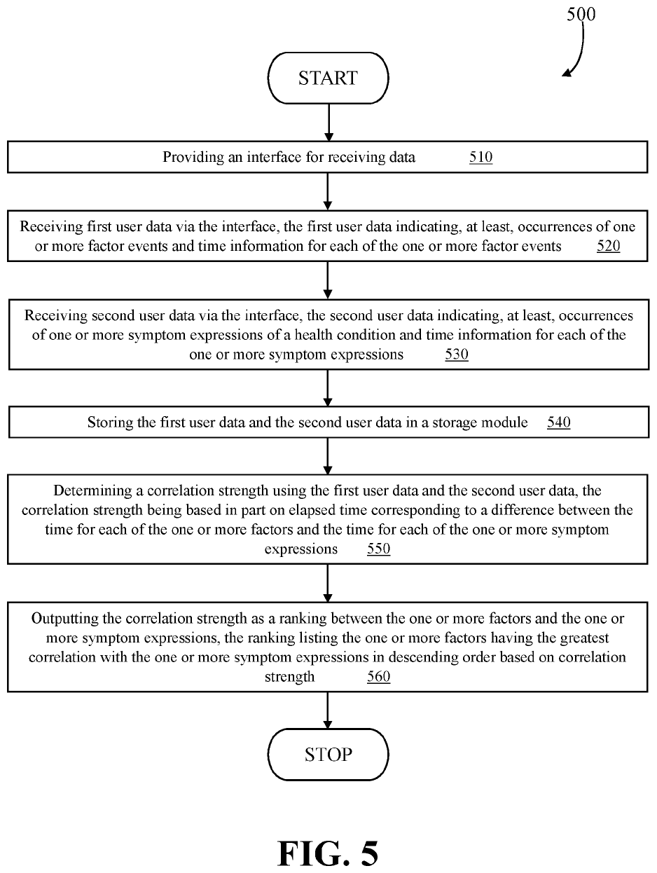

FIG. 5 illustrates a flow diagram of an embodiment of a method 500. The method 500 can be used, for instance, to determine a strength of a correlation between at least one potential trigger event and a subsequently occurring episodic condition. In one example, the method 500 can be carried out by non-transitory computer-executable instructions that are executed by programmable processing circuitry, for instance, including use of a user interface at a computing device as described elsewhere herein.

At 510, the method 500 includes providing an interface for receiving data. Interface may be a user interface of an electronic device such as user interface 100 or user interface 300. For example, user interface may include one or more regions where a user can input factor and/or symptom expression event time information, or where a user can set up a process for recording factor event and/or symptom expression time information. Such a process could be an input element (e.g., time selector for when a workout began, or when someone had coffee) or a permission to record data element, such as user interface 400 (e.g., “Would you like this application to record barometric pressure drops in your area?”; “Would you like this application to track your location”; “Would you like this application to track your screen time?”). Data can also be automatically received by the user interface from a database or other electronic/digital source where data is stored and/or recorded (e.g., a smartphone can automatically track time which can be integrated with the interface).

At 520, the method 500 includes receiving first user data via the interface, the first user data indicating, at least, occurrence of one or more factor events (i.e., potential trigger events) and time information for each of the one or more factor events. The factor can be a potential triggering event for a subsequent episodic condition (i.e., has an effect on the body) or can have no apparent effect of creating a subsequent episodic condition (i.e., has no effect on the body). First user data can be manually entered by a user or another individual, or automatically received by the interface from a database or other electronic/digital source. In additional embodiments, the factor at 510 can be a first potential trigger event, and the method 500 can additionally include receiving user input indicating another time (e.g., a third time) that is associated with an occurrence of a second factor.

First user data, such as the first potential triggering event and/or the second potential triggering event, as well as any subsequent data received, can include, as non-limiting examples, information relating to one or more of: a type of food and/or beverage consumed (e.g., coffee, caffeine, chocolate, alcohol, cheese), a relative quantity corresponding to the triggering event (e.g., three cups of coffee, two bananas), exposure to a type of ambient condition (e.g., exposure to an airborne particulate, such as an allergy-symptom inducing airborne particulate, such as pollen), a type of medication taken, a type of therapeutic treatment received, a type of result of a medical diagnostic test, a status of, and/or change in, an anatomic characteristic of a user (e.g., anatomic characteristics that can change over time, such as body temperature and/or pupil size; anatomic characteristics that generally do not change naturally over a certain period of time, such as hair color, height, Epstein-Barr status, and/or gender), a user's mood, a user's menstrual cycle, a user's overall “day” score, a user's overall pain level, a user's plane flight(s), a user's physical location(s), weather (e.g., rain, snow, barometric pressure, air quality), a user's physical exercise (e.g., type of workout), a user's time at an imaging screen, a user's duration of time working, and a user's sleep information.

In addition to receiving both a type of potential trigger event and time information of the occurrence of at least one potential triggering event, information received in relation to the potential triggering event can include one or more of: information relating to an amount, or volume, of a particular type of potential triggering event (e.g., location, such as zip code, at which the triggering event occurred), information relating to one or more user attributes (e.g., at the time the triggering event occurred, for instance, zip code, number of children, maternal haplogroup), information relating to one or more characteristics of other users (e.g., using information of users with one or more similar profile characteristics, or information on events received from users with one or more similar profile characteristics), information relating to a diagnosis or a self-diagnosis, information that was collected automatically rather than user manual entry (e.g., barometric pressure changes entered into the user's log by an API based on user zip code), and information relating to any type of event the user chooses to specify.

At 530, the method 500 includes receiving second user data via the interface, the second user data indicating, at least, occurrence of one or more symptom expressions of a health condition (i.e., episodic condition) and time information for each of the one or more symptom expressions. Second user data can be manually entered by a user or another individual, or automatically received by the interface from a database or other electronic/digital source. The type of episodic condition can be, for example, the occurrence of an episode of a chronic condition (e.g., occurrence of a migraine) or one or more symptoms associated with the occurrence of an episode of a chronic condition (e.g., nausea, aura, head pain, sinus congestion, etc.) (e.g., a plurality of related symptoms, such as fatigue, head pain, and muscle soreness). The type of episodic condition can be the occurrence of a health condition or two or more health conditions simultaneously. In embodiments, the second user data can be received and stored together with the first user data (e.g., within seconds, minutes, or another short time interval), or the second user data can be received and stored separate from the first user data (e.g., over days, weeks, or another long time interval), either before or after the first user data is received and/or stored.

The time information received in association with each of the occurrence of a type of potential trigger event and occurrence of a type of episodic condition can be received in any of a variety of forms. For example, the time information received can be a start time (e.g., started a type of exercise routine), an end time (e.g., finished eating a banana), a point in time between the start time and the end time (e.g., was eating a banana at this time), a timespan (e.g., 45 minute exercise routine), and/or a descriptor of a general time of a day (e.g., receiving time as “morning,” “afternoon,” or “evening”). As a further example, the time information received in association with each of the occurrence of a type of potential trigger event and occurrence of a type of episodic condition can be received in various increments and durations and can be a discrete point in time (e.g., 8:01 am) or a duration of time (e.g., measured in seconds, minutes, hours, days, or weeks) (e.g., being in a semester of college vs. on a break could be used as a received time associated with a type of potential trigger event—being an active student—and used, for instance, in compound trigger event finding; for example, a user might have triggering events in the form of stressors that happened only while in school). As another example, for a user who may experience a generally constant presence of an episodic condition, the received time information associated with the occurrence of a type of episodic condition can be a time associated with an increase or decrease in the severity of that episodic condition.

At 540, the method 500 includes storing the first user data and the second user data in a storage unit. The storage unit may be physically located on a computing device, such as a mobile computing device or another similar electronic device, or digitally located in a remote location, such as a cloud server. A user may access the storage unit via user interface 100, user interface 300, or another suitable interface or device known to one of ordinary skill in the art. The storage unit may be configured to store other data and information such as environment data, third user data, fourth user data, and so on. The storage unit may be unique to a single user or shared among a community of users in a shared space.

At 550, the method 500 includes determining correlation strength using the first user data and the second user data, the strength of correlation being based on the time information for each of the one or more factor events and the time information for each of the one or more symptom expressions. In a further embodiment of the method 500 where times associated with more than one potential triggering event are received, the method can include determining correlation strength between each further potential triggering event and each further occurrence of a type of episodic condition. In addition to using the elapsed time between each potential triggering event and a subsequently occurring type of episodic condition, the certain embodiments of the method 500 could include using an end time of the type of episodic condition along with a type of remediating action taken to assess the efficacy of that remediating action to address the episodic condition (e.g., using an end time of a head pain/migraine episode to evaluate a remediating correlation of taking a type of medication, such as Aleve, impacts the reduction of migraine episode for that particular user). In embodiments, correlation strength may be determined using an evaluation system.

In some cases, if multiple instances of the same type of potential triggering condition are received prior to occurrence of an episodic condition, an average time can be used. For instance, in the example of a migraine episodic condition, migraine severity can change over time, and a weighted midpoint of a migraine episode, based on migraine severity changes, could be more accurate than using a start time when scoring and/or ranking of potential triggering event correlations to the occurrence of the subsequent episodic condition. In another instance, again in the example of a migraine episodic condition, migraine prodrome can include a variation of types of events and severity, and, thus, the start time may be extrapolated from other time-based datapoints rather than being a start time that is received directly from the user.

At 560, the method 500 includes outputting the correlation strength to the interface. In embodiments, the correlation strength may be outputted as a ranking between the one or more factor events and the one or more symptom expressions, the ranking listing the one or more factor events having the greatest correlation with the one or more symptom expressions in descending order based on correlation strength. In a further embodiment of the method 500 where times associated with more than one potential triggering event are received, the method can include outputting the correlation strength as a ranking for each further potential trigger event and each further subsequent occurrence of the episodic condition to determine a ranking (e.g., relevance score) for each such potential trigger event to the occurrence of the episodic condition. For example, using the elapsed time to determine the relevance score of the potential trigger event to the occurrence of the episodic condition can include the relevance score being an indication of a strength of a correlation between the potential trigger event and the occurrence of the episodic condition.

The correlation determined, for example using the elapsed time between one or more potential triggering events and the subsequently occurring episode of an episodic condition, can be in a variety of forms. As an example, determining such correlation can include one or more of: determining an increase or decrease in the occurrence of the episodic condition (e.g., eating bananas makes migraines become more or less frequent), determining a change in frequency of episodes of the episodic condition (e.g., less frequent migraines), determining a change in the severity of one or more episodes of the episodic condition (e.g., less pain overall in a migraine episode), determining one or more clusters of related potential triggering events occurring within a predetermined time range of one another, determining a time span between two or more different potential triggering events and then determining a correlation between the time span between these two or more different potential triggering events and the time it takes for the subsequent occurrence of an episode of an episodic condition (e.g., eating a banana within a certain time of eating strawberries then produces a subsequent migraine that is more severe than a migraine resulting from only one of bananas and strawberries or eating bananas and strawberries at more spaced apart time instances), determining a correlation between amounts of any ingredient/component of an intake potential triggering event (e.g., an amount of caffeine in a beverage being greater than a preset threshold; tyramine; histamine; etc.), and determining a correlation between two of the same types of potential triggering events (e.g., different pairs of eyeglasses with specific pair causing an increase in migraine episodes).

In some embodiments, in addition to using the elapsed time between one or more potential triggering events and the subsequently occurring episode of an episodic condition, other factors can be used to determine a relevance score of the potential triggering event to the occurrence of the episodic condition. As an example, such other factors can include one or more of: which episode side effects are more associated with each medication, which medications work better to reduce subsequent episodes when taken together, which symptom expressions correlate more to certain potential triggering events (e.g., when right side head pain, these potential triggering events have a high correlation, but when head pain is all over, these other potential triggering events have a high correlation), and whether a particular treatment delays onset of one or more symptoms (e.g., speed to effectiveness vs. absolute remediation of episode).

Examples of episodes of episodic conditions have been used herein, including migraines, though it is to be understood that the disclosure herein can be applied to a variety of other types of health conditions. As one example, the use of an elapsed time between one or more potential triggering events and a subsequently occurring health condition can be applied in diabetes management. For instance, this can include using timing-based determinations to score changes in a test result status for a diabetes patient. In one particular example, a group of the potential triggering events of eating a donut could result in determining a correlation to a lower A1C level as a type of bodily change resulting from the group of the potential triggering event.

In addition to, or alternative to, the above disclosed features, embodiments within the scope of the present disclosure can include one or more of the following features.

It could be possible that a potential triggering event correlates with both an actual trigger of the episode and the episode itself, but yet that potential triggering event is not actually a trigger itself and, thus, could be a false positive. In such an instance, removing this potential triggering event could reveal a pattern in the onset of episodes (e.g., hormones can affect migraines, and when comparing bananas to migraines, it may be only that potential triggering events on the user's menstruation days are used in the technique to determine a correlation, and doing so can remove false positive migraines as falsely associated with banana intake when in fact it is more likely to be caused by menstrual related factor(s)).

In some examples, the techniques disclosed herein can utilize one or more variations of cohort analysis that includes pooled data from multiple users. Using the timing-based patterns to find factors from a plurality of user's data could be part of finding correlation strength in an increase or decrease between certain months or years for external factors (e.g., pollen was high a certain month, and cohort analysis can show that a plurality of users had a 13% increase in reported migraines compared to earlier time periods).

It can be possible that some potential triggering events, when occurring within a predetermined time period of one another, can compound symptoms/occurrences of an episode as compared to these same potential trigger events in isolation. For example, a user may have more migraines on a certain day of the week when that user visits a particular location (e.g., a certain yoga studio), but not as much on the other days of the week or when visiting different locations (e.g., other yoga studios).

In embodiments, method 500 may further include outputting the correlation strength as a ranking between the one or more factor events and the one or more symptom expressions of a health condition, the ranking listing the one or more factor events having the greatest correlation with the one or more symptom expressions of a health condition in descending order based on correlation strength. In embodiments, method 500 may further include one or more of receiving third user data via the interface, the third user data indicating, at least, occurrence of one or more factor events and time information for each of the one or more factor events; receiving fourth user data via the interface, the fourth user data indicating, at least, occurrence of one or more symptom expressions of a health condition and time information for each of the one or more symptom expressions of a health condition; storing the third user data and the fourth user data in the storage unit; determining an updated correlation strength using the first user data, the second user data, the third user data, and the fourth user data; and outputting the updated correlation strength to the interface. In embodiments, first user data and second user data may be received from a first user while third user data and fourth user data are received from a second, different user. Accordingly, method 500 may include receiving data from multiple users or sources to form a population data pool which can be used, for example by the evaluation system, to determine correlation strength based on past user experiences and correlations in addition to the received first and second user data from a first, present user.

In embodiments, method 500 may further include one or more of receiving environment data indicating, at least, weather conditions corresponding to a predetermined area; storing the environment data in the storage unit; determining an updated correlation strength using the first user data, the second user data, and the environment data; and outputting the updated correlation strength to the interface, optionally as a ranking between the one or more factor events and the one or more symptom expressions of a health condition. Environment data may be received directly from a user or automatically from an environment data tracking application configured to send environment data to other sources. In embodiments, the environment data may be sent directly to the storage unit which can be coupled to a processing unit configured to perform, among other things, the operations of determining correlation strength and outputting the correlation strength to an interface.

In embodiments, method 500 may further include sending a treatment recommendation based on the correlation strength. The treatment recommendation may be a medication, a suggestion to exclude or limit a certain factor entirely or at certain times, a suggestion to visit a doctor, or reference to one or more external sources that can provide more information to the user. In embodiments, method 500 may further include one or more of receiving treatment data corresponding to the outcome or effectiveness of a treatment recommendation; determining a treatment correlation using the treatment data, the treatment correlation being based on the outcome or effectiveness of the treatment recommendation as compared to one or more subsequent symptom expressions of a health condition; outputting the treatment correlation; and displaying the treatment correlation on the user interface and storing the treatment correlation in the storage unit.

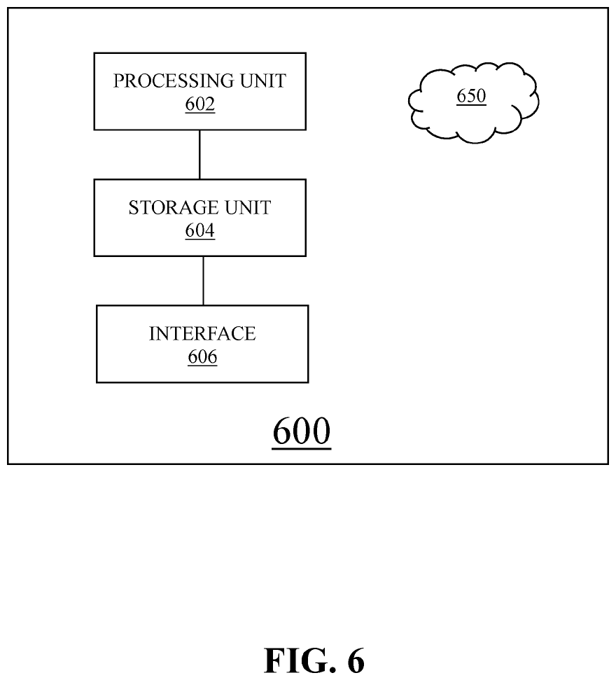

FIG. 6 illustrates an embodiment of a system 600 for carrying out a method of determining correlation strength between at least one potential trigger event and a subsequently occurring episodic condition. System 600 may include at least one processing unit 602, at least one storage unit 604, and at least one interface 606 which may be user interface 100 or user interface 300. Processing unit 602 may be communicatively couplable to both storage unit 604 and interface 606 such that data can be freely transmitted across system 600. Interface 606 may couple to any one or more of several interface devices, including a network connection, a display such as a computer monitor, and/or a keyboard which can be used by a user to effectuate inputting and outputting of data from system 600. In embodiments, storage unit 604 may be couplable to a digital storage 650, such as a cloud server or other digital storage unit, for storing data externally.

Processing unit 602 and storage unit 604 are configured to perform operations for the method of determining correlation strength, including the operations of method 500 such as receiving first and second user data, storing the first and second user data, determining correlation strength using the received data, outputting the correlation strength to a user interface, displaying the correlation strength on the user interface and storing the correlation strength, receiving third and fourth user data, storing the third and fourth user data, receiving environment data, storing the environment data, determining an updated correlation strength using the received data, and outputting an updated correlation strength.

Embodiments of the disclosure include a non-transitory storage article for performing an evaluation of correlation strength between one or more factor events and one or more symptom expressions of a health condition, including non-transitory computer executable instructions for performing operations, including the operations of method 500 such as providing an interface for receiving data, optionally from a user, receiving first and second user data via the interface, storing the first and second user data, determining correlation strength using the received data, outputting the correlation strength to the interface, displaying the ranking on the interface and storing the correlation strength, receiving third and fourth user data, storing the third and fourth user data, receiving environment data, storing the environment data, determining an updated correlation strength using the received data, and outputting an updated correlation strength.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations, and locations, etc. have been described for use with disclosed embodiments, others besides those disclosed may be utilized without exceeding the scope of the claimed inventions.

It should be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more processing units, one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structures or any other physical structure suitable for implementation of the described techniques. Also, the techniques could be fully implemented in one or more circuits or logic elements.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

For purposes of interpreting the claims, it is expressly intended that the provisions of 35 U.S.C. § 112(f) are not to be invoked unless the specific terms “means for” or “step for” are recited in a claim.

1. A method performing operations stored in a non-transitory computer readable medium, the operations comprising:
providing an interface for receiving data;
receiving first user data via the interface, the first user data indicating, at least, occurrence of one or more factor events and time information for each of the one or more factor events;
receiving second user data via the interface, the second user data indicating, at least, occurrence of one or more symptom expressions of a health condition and time information for each of the one or more symptom expressions of a health condition;
storing the first user data and the second user data in a storage unit;
determining correlation strength using the first user data and the second user data, the correlation strength being based on the received time information for each of the one or more factor events and the received time information for each of the one or more symptom expressions of a health condition; and
outputting the correlation strength to the interface,
wherein the first user data and the second user data can be received and stored separately in any order or received and stored together.

2. The method of claim 1, wherein the interface is a user interface of an electronic device, the user interface configured for receiving data directly from a user.

3. The method of claim 1, further comprising:
receiving third user data via the interface, the third user data indicating, at least, occurrence of one or more factor events and time information for each of the one or more factor events;
receiving fourth user data via the interface, the fourth user data indicating, at least, occurrence of one or more symptom expressions of a health condition and time information for each of the one or more symptom expressions of a health condition;
storing the third user data and the fourth user data in the storage unit;
determining an updated correlation strength using the first user data, the second user data, the third user data, and the fourth user data; and
outputting the updated correlation strength to the interface.

4. The method of claim 3, wherein the first user data and the second user data are received from a first user, and the third user data and the fourth user data are received from a second user.

5. The method of claim 1, wherein the correlation strength is outputted to the interface as a ranking between the one or more factor events and the one or more symptom expressions of a health condition, the ranking listing the one or more factor events having the greatest correlation with the one or more symptom expressions of a health condition in descending order based on correlation strength.

6. The method of claim 1, wherein the first user data indicates quantity information used in determining correlation strength.

7. The method of claim 1, wherein the time information for each of the one or more factor events and the time information for each of the one or more symptom expressions of a health condition each comprise a start time and an end time.

8. The method of claim 1, wherein the time information for each of the one or more factor events and the time information for each of the one or more symptom expressions of a health condition each comprise a descriptor of a general time of day.

9. The method of claim 1, further comprising:
receiving environment data indicating, at least, weather conditions corresponding to a predetermined area;
storing the environment data in the storage unit;
determining an updated correlation strength using the first user data, the second user data, and the environment data; and
outputting the updated correlation strength to the interface.

10. The method of claim 1, wherein the first user data includes at least one of (i) consuming food or drinks, (ii) ingesting a medication, or (iii) participating in a physical activity.

11. The method of claim 1, wherein the first user data includes an indication of the absence of at least one of (i) consuming food or drinks, (ii) ingesting a medication, or (iii) participating in a physical activity.

12. The method of claim 1, wherein the interface includes one or more regions configured to receive time information for the one or more factor events and time information for the one or more symptom expressions.

13. The method of claim 1, wherein the correlation strength is based on an elapsed time determined explicitly using an algorithm that calculates a difference between the time information for each of the one or more factor events and the time information for each of the one or more symptom expressions of a health condition.

14. The method of claim 1, wherein the correlation strength is based on an elapsed time determined implicitly from a table of values corresponding to the time information for each of the one or more factor events and the time information for each of the one or more symptom expressions of a health condition.

15. The method of claim 1, further comprising outputting a treatment recommendation based on the determined correlation strength.

16. A system comprising: a user interface; and
a processing unit and a storage unit, the processing unit and the storage unit configured to perform operations comprising:
receiving first user data via the user interface, the first user data indicating, at least, occurrence of one or more factor events and time information for each of the one or more factor events;
receiving second user data via the user interface, the second user data indicating, at least, occurrence of one or more symptom expressions of a health condition and time information for each of the one or more symptom expressions of a health condition;
storing the first user data and the second user data in a storage unit;
determining correlation strength using the first user data and the second user data, the correlation strength being based on the received time information for each of the one or more factor events and the received time information for each of the one or more symptom expressions of a health condition; and
outputting the correlation strength to the interface,
wherein the first user data and the second user data can be received and stored separately in any order or received and stored together.

17. The system of claim 16, wherein the correlation strength is outputted to the user interface as a ranking between the one or more factor events and the one or more symptom expressions of a health condition, the ranking listing the one or more factor events having the greatest correlation with the one or more symptom expressions of a health condition in descending order based on correlation strength.

18. The system of claim 16, wherein the processing unit and the storage unit are further configured to perform the operations of:
receiving third user data via the user interface, the third user data indicating, at least, occurrence of one or more factor events and time information for each of the one or more factor events;
receiving fourth user data via the user interface, the fourth user data indicating, at least, occurrence of one or more symptom expressions of a health condition and time information for each of the one or more symptom expressions of a health condition;
storing the third user data and the fourth user data in the storage unit;
determining an updated correlation strength using the first user data, the second user data, the third user data, and the fourth user data; and
outputting the updated correlation strength to the user interface.

19. The system of claim 16, wherein the processing unit and the storage unit are further configured to perform the operations of:
receiving environment data indicating, at least, weather conditions corresponding to a predetermined area;
storing the environment data in the storage unit;
determining an updated correlation strength using the first user data, the second user data, and the environment data; and
outputting the updated correlation strength to the user interface.

20. A non-transitory storage article for performing an evaluation of correlation strength between one or more factor events and one or more symptom expressions of a health condition, including non-transitory computer-executable instructions for:
providing an interface for receiving data;
receiving first user data via the interface, the first user data indicating, at least, occurrence of one or more factor events and time information for each of the one or more factor events;
receiving second user data via the interface, the second user data indicating, at least, occurrence of one or more symptom expressions of a health condition and time information for each of the one or more symptom expressions of a health condition;
storing the first user data and the second user data in a storage unit;
determining correlation strength using the first user data and the second user data, the correlation strength being based on the received time information for each of the one or more factor events and the received time information for each of the one or more symptom expressions of a health condition; and
outputting the correlation strength to the interface,
wherein the first user data and the second user data can be received and stored separately in any order or received and stored together.
